(19) United States
(12) Patent Application Publication
(10) Pub. No.: US 2011/0104286 A1
(43) Pub. Date: May 5, 2011

(54) PHARMACEUTICAL COMPOSITIONS CONTAINING THE ENZYME CYPROSIN, AN ASPARTIC PEPTIDASE FROM CYNARA CARDUNCULUS AND ITS INCLUSION IN ANTITUMOUR FORMULATIONS

(76) Inventors: Maria Salomé Soares Pais, Lisboa (PT); Pedro Nuno De Sousa Sampaio, Forte da Casa (PT); Rita Isabel Ganchas Soares, Oeiras (PT); Maria Constanca Baptista Coelho, Oeiras (PT); Jorge Miguel Silva Santos, Oeiras (PT); Pedro Estilita Monteiro Da Cruz, Oeiras (PT); Helder Joaquim Soares Da Cruz, Oeiras (PT)

(21) Appl. No.: 12/680,530

(22) PCT Filed: Nov. 28, 2008
(86) PCT No.: PCT/IB2008/055009

§ 371 Date: Nov. 1, 2010

(30) Foreign Application Priority Data
Sep. 28, 2007 (PT) 103839

(51) Int. Cl. A61K 38/48; C12N 9/50; C12N 9/96; A61K 9/14; A61P 35/00; A61P 1/00; A61P 31/14; A61P 9/00; B82Y 5/00

(52) U.S. Cl. 424/489; 435/219; 424/94.66; 435/188; 977/773; 977/906

(57) ABSTRACT

An aspect of the present invention is the use of a preparation containing a phytepsin, more specifically a cyprosin, containing the heterodimer, its N-terminal pro-peptide, the mature N-terminal peptide, and mature C-terminal peptide, as well as other precursor species, processing products, and aggregate species, either isolated or in any combinations of the former, native, extracted and partially purified from flowers of Cynara cardunculus, or recombinant, extracted from the supernatant from a culture of Saccharomyces cereviseae genetically modified for the heterologous production of cyprosin, for therapeutic applications more precisely for its use as an antitumor agent.

### TECHNICAL FIELD OF THE INVENTION

An aspect of this invention is the development of pharmaceutical formulations containing a preparation of a phytepsin, more specifically a cyprosin, characterized as being an aspartic protease native from Cynara cardunculus flowers (Access number at UniProtKB/TrEMBL: Q39476).

An aspect of the present invention is a preparation of the referred cyprosin containing the heterodimer, the cyprosin pre-propeptide and/or the cyprosin propeptide containing the N-terminal and/or the lobe/chain/N-terminal mature subunit and/or the cyprosin propeptide containing the C-terminal and/or the PSI domain, specific of plant phytepsins and/or the lobe/polypeptide chain/N-terminal mature subunit and/or the isolated polypeptide containing the PSI domain or any other secondary product derived from processing or degradation of the initial pre-propeptide as well as other precursor species, processing products and aggregate species, either isolated or under any combination of the former.

An aspect of this invention is a preparation of either native cyprosin, extracted from flowers of Cynara cardunculus, or recombinant cyprosin, extracted from a supernatant resulting from the culture of a Saccharomyces cerevisiae genetically modified for the production of the heterologous protein.

It is an aspect of the present invention the inclusion of a preparation containing the referred cyprosin in pharmaceutical formulations with antitumour activity demonstrated in vitro in human epithelial cell lines, namely a colon derived cell line (HCT), an adenocarcinoma-derived cell line (HeLa), a fibrosarcoma-derived cell line (HT) and a rabdomyosarcoma-derived cell line (TE).

### BACKGROUND OF THE INVENTION

The mechanisms of multiplication and aging of normal (non-tumour) cells and those of tumour cells are similar. The anomalous regulation of one of these mechanisms may induce tumour formation. Factors such as chemical or radiation agents can damage DNA and alter the expression of genes involved in programmed cell death (PCD) or apoptosis, giving rise to uncontrolled cell proliferation in the absence of growth factors.

Proteolytic enzymes, named as peptidases, proteases, or proteinases, hydrolyze peptide bonds. Exo-peptidases act near the terminal polypeptide region while endo-peptidases cleave the polypeptide chain internally with higher or lower specificity, depending on the nature of the enzyme. Endo-peptidases play an important role in the transmission of biochemical signals required to the correct function of PCD programs. According to their catalytic mechanism, endo-peptidases are divided in 5 distinct sub-classes: serine peptidases, cystein peptidases, aspartic peptidases, threonin peptidases and metalopeptidases (Rawlings and Barret, 1999; Beers et al., 2000). The processes involved in PCD occur in three different and linked pathways: synthesis and emission of induction signals (extracellular); transmission of induction signals (intracellular) and finally, an intracellular pathway common to all cells, termed execution pathway (Roberts et al., 1999). Endo-peptidases generate specific signals for induction of PCD by processing and delivery of bioactive molecules and activation of receptors at the cell surface [e.g. cytokines TNF-α, γ interferon (IFN-γ), TGF-β, and the receptor ligand for Fas/APO-1] (Deiss et al., 1996).

The role of endo-peptidases, namely caspases, on transmitting inducing PCD signals is widely documented. Caspases, for example, through cleavage and consequent inhibition of endo-nuclease inhibitor proteins, indirectly promote cleavage of nuclear DNA. This explains the morphological alterations observed in cells entering apoptosis, namely the decrease in size and the condensation of the cell nucleus (Muzzio, 1998; Horta, 1999).

In what the execution pathway is concerned, proteases may act by processing/cleaving two types of molecules, from two distinct functional groups: molecules involved on the organization and maintenance of the cellular structure and enzymes involved on homeostasis (Thornberry et al., 1997).

Louis Deiss et al. (1996), using a random gene silencing approach by antisense cDNA, prepared from cells exposed to cytokines, showed that the anti-sense RNA from the aspartic protease Cathepsin D (CatD) was able to protect a human epithelial cell line, derived from an adenocarcinoma (HeLa cells), from PCD via IFN-γ, Fas/APO-1 and TNF-α (Deiss et al., 1996). This was the first among many studies that revealed the direct role of CatD on the induction of programmed cell death mediated or not by cytokines. Since then, many other mechanisms have been suggested to explain the function of CatD in the induction of PCD. Wu et al. (1998) pointed out a role of CatD on the suppression of tumours depending on factor p53. Later on, Bidere et al. (2003) suggested that induction of the apoptosis phenotype of human T-lymphocytes via CatD results from the inactivation of the Bax protein that induces the selective release of factor AIF (a mitochondrial protein), functioning specifically as an activator of the apoptosis initiation process. According to Piwnica et al. (2004), CatD was able to process human prolactin giving rise to small fragments similar to its N-terminal. Do to its angiogenic activity these fragments play an inhibitory role on tumour development. In the same year Iacobuzio-Donahue et al. studied the expression pattern of CatD using western blotting, immuno-hystochemistry and glycosilation analysis techniques in 59 samples of colon tumour. By examining the content and the expression of CatD, those authors were able to correlate the loss of CatD expression with pathology in more than 50% of the observed samples. Later on, Haendeler et al (2005) reported on the role of cathepsin D on PCD via degradation of Tioredoxine-1 (Trx), an essential anti-apoptotic protein derived from its capacity to sequester reactive oxygen radicals (ROR). More recently, it has been demonstrated that CatD stimulates caspase-dependent apoptosis in a rat tumour embryonic cell line (line 3Y1-Ad12) and in human chronic myelogenic leukaemia (K562). In the first case, CatD-mediated apoptosis is independent of its catalytic activity which accounts for the relationship with structural features (Beaujouin et al., 2006; Wang et al., 2006).

Presently, a primordial role of CatD in animal cell apoptosis can not be foreseen, and it is not possible to conclude if its apoptotic activity is due to a single mechanism. The final effect may be due to several interlinked pathways that may be explored in order to find new agents/molecules against some cancer types.

Contrasting to the knowledge existing on PCD in animal models, there are no reports on a direct relationship between a peptidase and PCD in plant cells. Dunn (2002) has reported on the mechanism beyond plant peptidases involvement in PCD in plants, drawing an analogy with animal cells. An example of plant peptidases with special relevance for the present innovation is that of phytepsins: aspartic pepsin-like endo-peptidases, family A1 (Beers et al., 2000). Phytepsins are the only aspartic endo-peptidases listed in the MEROPS database (a reference database of peptidases and corresponding specific inhibitors) described as being related to PCD in plants (Rawlings et al., 2006). Evidence for that assumption is that the levels of mRNA expression of these enzymes increase in leaves and petals along senescence (Buchanan-Wollaston, 1997; Panavas et al., 1999). Phytepsins are synthesized as pre-pro-peptides with high homology with animal CatD, with exception for 100 residues near the C-terminal designated by PSI (plant specific insert) domain. As the name suggests, this domain is specific for phytepsins (Runeberg-Roos et al., 1991). The PSI domain presents high homology with saposins (enzymes known to be activators of sphyngolipids in animals). The PSI domain is separated from the pro-peptide C-terminal by a cleavage, occurring during post-translation processing, which cuts the pro-peptide into two nearly equivalent portions (Ramalho-Santos et al., 1998). This initial cleavage can be auto-catalytic, as it happens with wheat phythepsin, and it is a requisite for the endo-peptidase activity of the mature protein.

In turn, the mature protein results from the assemblage of two chains: one heavier chain, derived from the processing of the N-terminal pro-peptide, resulting from the first cleavage; and a lighter chain, consisting of the C-terminal of the second portion, containing the PSI domain. Due to the absence of the PSI domain, the N-terminal pro-peptide presents a typical structure common all pro-forms of the animal and microbial aspartic endo-peptidases, such as CatD (Ramalho-Santos et al., 1998).

A typical case of phythepsins with high homology with CatD is the cyprosin family, formerly designated by cynarases, or cinarins, that have been isolated for the first time by Heimgartner et al. (1989) from the flowers of Cynara cardunculus (thistle). Just like cardosins, traditionally used for producing cheese in the Iberian Peninsula, cyprosins have been described for the first time as being aspartic endo-peptidases, heterodimeric, glycosylated, with maximal activity at pH 5.1, when using casein as substrate (Cordeiro et al., 1994). Since then, a cDNA library was constructed and a clone containing the cDNA coding for cyprosin 3 and the sequence of CYPRO11 gene was deciphered. These results have being followed by cyprosin 3 characterization and its hystochemical localization within the different organs of the C. cardunculus flower was studied (Cordeiro et al., 1994; 1995; 1998; Brodelius et al., 1995; 1998). More recently, other studies have been performed revealing not only the global structure of these proteases (the sequences of their pre- and pro-domains), their glycosylation patterns, as well as their typical processing mechanism (Faro et al., 1995, Verissimo et al., 1996; Costa et al., 1997; Ramalho-Santos et al., 1997; 1998; Bento et al., 1998; Frazão et al., 1999).

Finally, the growing economic and therapeutic interest of aspartic endo-peptidases have triggered the expression of CYPRO11 gene in yeast aiming at the large scale production of cyprosins for industrial applications (Pais et al., 2000; WO1196542).

### BRIEF DESCRIPTION OF THE DRAWINGS

FIG. 1: Culture of control non-tumour cells FHs74 Int and culture of tumour cells HCT. A—FHs74 cells before addition of native cyprosin solution; B—FHs74 Int cells 48 hours after addition of 100 μg/mL of a native cyprosin solution; C—HCT cells before addition of native cyprosin solution of a native cyprosin preparation; D—Cells HCT 48 h after addition of 100 μl/mL. In contrast to FHs74 Int cells, that are not affected by native cyprosin addition, HCT cells present evidence of lysis 48 h after addition of the enzyme. Scale bar=100 μm.

FIG. 2: Cell viability evaluated by cell staining with SRB, plotted against the logarithm of the concentration (μg/ml) of native cyprosin tested for each of the tumour cell lines assayed: A—HCT, B—HT, C—TE, D—Hela.

FIG. 3: Viability of cells evaluated by cell staining with SBR, plotted against the logarithm of the concentration (μg/ml) of native cyprosin for each of the non-tumour cell lines tested A—Vero Cells, B—FHs74 Int Cells.

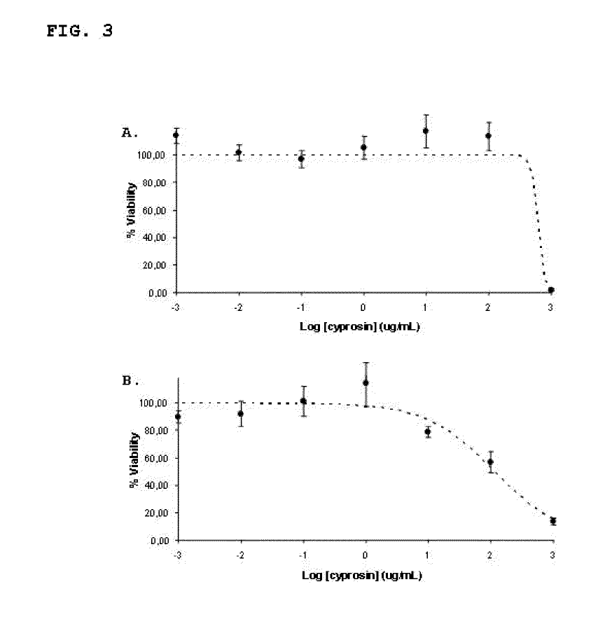

FIG. 4: Control non-tumour cells FH74 Int and tumour cells HCT. A—FHs74 Int cells before addition of recombinant cyprosin; B—FHs74 Int cells 48 h after addition of 100 μg/mL recombinant cyprosin preparation; C—HCT cells before addition of recombinant cyprosin preparation; D—HCT cells 48 h after addition of 100 μg/mL recombinant cyprosin preparation. In contrast to FHs74 Int cells, showing no effects addition of recombinant cyprosin, HCT cells present clear evidence of lysis 48 h after addition of recombinant cyprosin preparation. Scale bar=100 μm

FIG. 5: Representation of cell viability by cell staining with SBR, plotted against the logarithm of the concentration (μg/ml) of recombinant cyprosin for each of the cells lines assayed. A—HCT tumour cells; B—FHs74 Int non-tumour cells.

### GENERAL DESCRIPTION OF THE INVENTION

The present invention is based on the cytotoxicity study of native and recombinant cyprosin preparations (Access number at UniProtKB/TrEMBL: Q39476), extracted either from Cynara cardunculus flowers or from the supernatant of a recombinant Saccharomyces cerevisiae culture (BJ1991), respectively.

The enzyme preparations contain both structural polypeptide chains: N-terminal chain (can be the N-terminal pro-peptide or the N-terminal mature peptide, or even a combination of both) and the C-terminal chain (mature C-terminal peptide).

Both native and the recombinant proteins were extracted and purified according to methods previously described (Brodelius et al., 1995; Pais et al., 2000).

The cytotoxicity study that originated the invention was performed using the method of sulforhodamine B (SRB). This method is a rapid and accurate method for measuring the cytotoxicity of a product by colorimetric quantification of the total cellular protein biomass in cultured human cell lines coloured with SRB. Under acidic conditions SRB links to the amino acids of basic proteins in cells previously fixed with trichloroacetic acid (TCA), indicating a total protein contents in the fixed cells that is proportional to the cell density in the culture plate. As a result, the increase or decrease in cell number in the culture plate results on a proportional alteration of the stain amount measured, which in turn is indicative of the cytotoxic effect of the compound under study (Skehan, et al., 1989).

The SRB amount is measured by its capacity of absorbing light at wave length of 565 nm. Using this method it is possible to evaluate the relative growth/viability of cells treated with the compound under study against control cells grown under the same conditions (Monks, et al., 1991).

The cyprosin cytotoxic effect was evaluated using human tumour and non-tumour cell lines by cell morphology observation and determination of the corresponding IC₅₀ in vitro (parameter indicating the cyprosin concentration at which cell proliferation is inhibited by 50%).

When comparing the results obtained for the effect of cyprosin on tumour versus non-tumour cell lines, it has been verified that for a concentration of 100 μg/mL the enzyme induced morphological alterations in all tumour cell lines assayed, accompanied by lysis. In turn, the non-tumour cell lines, submitted to the same (100 μg/mL) cyprosin concentration, showed no significant alterations in morphology and cell growth. When the IC₅₀ values for the cyprosin effect in tumour cell lines was compared with the IC₅₀ values obtained with non-tumour cell lines, it has been observed that, in general, the enzyme preparations showed a higher lethal effect on tumour cell lines, without affecting the viability/growth of non-tumour cell lines significantly.

### DETAILED DESCRIPTION OF THE INVENTION

Due to the nature of this invention its detailed description is better achieved through examples.

The following examples illustrate the invention without limiting its scope.

### Example I

Anti-tumour activity of a preparation of native cyprosin containing both structural chains: N-terminal chain (consisting on the N-terminal pro-peptide and the mature N-terminal) and C-terminal chain (mature peptide C-terminal), isolated and purified from dried Cynara cardunculus flowers.

The cyprosin preparation was obtained from dried Cynara cardunculus flowers as previously described by Brodelius et al., 1995. The anti tumour activity of the enzyme preparation was evaluated using four human tumour cell lines: an epithelial cell line derived from a carcinoma (HCT116, ATCC CCL-247), an epithelial cell line derived from a fibrosarcoma (HT1080, ATCC CCL-121), an epithelial cell line derived from a rabdomyosarcoma (TE671, ATCC CCL-136), and an epithelial cell line derived from an adenocarcinoma (Hela, ATCC CCL-2™), and two non-tumour cell lines: one consisting of human intestinal (epithelial) cells (FHs74 Int, ATCC CCL-241) and another consisting of African green monkey kidney epithelial cells (Vero, ATCC CRL-1587).

The tumour cell lines HCT116, HT1080 e TE671 were inoculated on basal medium DMEM (Cambrex), supplemented with 5% Foetal Bovine Serum (FBS—Gibco). The final concentrations of glucose (Sigma) and of L-glutamine (Sigma) were of 4.5 g/L e 6.0 mM, respectively. The culture medium was supplemented with a 1% Penycillin/Streptomycin (Gibco) solution.

The tumour Hela cell line was inoculated on DMEM (Cambrex) basal medium, supplemented with 10% FBS (Gibco); 2.1 g/L sodium bicarbonate (NaHCO₃—Sigma); 1.0 mM sodium pyruvate (C₃H₃NaO₃—Sigma); and 0.1 mM of a non-essential amino acids solution (NEAA—Cambrex). The final concentrations of glucose (Sigma) and of L-glutamine (Sigma) were 1.0 g/L and 2.0 mM, respectively. The culture medium was supplemented with 1% Penycillin/Streptomycin (Gibco).

The non-tumour Vero cells were inoculated on basal medium DMEM (Cambrex), supplemented with 10% Foetal Bovine Serum (FBS—Gibco) and 3.56 mM L-glutamine (Sigma). The culture medium was also supplemented with 1% Penicillin/Streptomycin (Gibco).

The non-tumour cell line FHs74 Int was inoculated on Hybricare (ATCC; Cat. 46-X), supplemented with 10% Foetal Bovine Serum (FBS—Gibco); 2.1 g/L NaHCO₃ (Sigma) solution; 2.0 mM L-glutamine (Sigma) and 30 ng/mL epidermal growth factor (EGF—Sigma). The medium was supplemented with 1% Penicillin/Streptomycin (Gibco).

The cells were propagated in a static culture system operated in batch. The cell concentration and viability were evaluated using the Trypan blue exclusion method.

Table III presents the specific growth rate (μ) and the corresponding doubling time (DT) for each cell culture.

TABLE III
Specific growth rate (μ) and the corresponding
doubling time of the tumour and non-tumour cell lines used.

| | Cells | μ (h⁻¹) | DT (h) |
|---|---|---|---|
| Tumour cell | HCT116 | 0.031 (±0.001) | 23 |
| lines | HT1080 | 0.020 (±0.001) | 35 |
| | TE671 | 0.024 (±0.001) | 29 |
| | Hela | 0.031 (±0.001) | 22 |
| Non-tumour | Vero | 0.029 (±0.001) | 24 |
| cell lines | FHs74 Int | 0.0041 (±0.0005) | 169 |

The IC₅₀ values were determined for the different cultured cell lines using the sulforhodamine B (SRB) method.

A total volume of 100 μL from each cell line was inoculated in triplicate in 96 well plates. The corresponding densities were estimated based on the specific growth rate of each replicate in such a way that after 24 h of treatment the cell cultures presented approximately 50% confluence. Following this strategy, the inoculum densities obtained for HCT116 and Hela cells were 3.1×10⁴ cells/cm²; for HT1018 and Vero cells were 4×10³ cells/cm²; for TE671 cell line were 1.6×10⁴ cells/cm², and for FHs74 Int cell line were 2.5×10⁴ cells/cm².

The cultures were incubated during 24 h a 37° C., in a 7% CO₂ atmosphere and 90% humidity.

24 h after inoculation, 100 μL of the a cyprosin preparation were added to each well at decreasing concentrations: 1000 μg/mL; 100 μg/mL; 10 μg/mL; 1 μg/mL; 0.1 μg/mL; 0.01 μg/mL and 0.001 μg/mL, for IC₅₀ calculation.

The plates were incubated during 48 h at 37° C., in a 7% CO₂ atmosphere and 90% humidity.

Control assays were performed for all cell lines used in the absence of cyprosin preparations.

48 h after addition of the enzyme preparation the cell cultures were observed under the light microscope to register the confluence and the morphological characteristics of the cells.

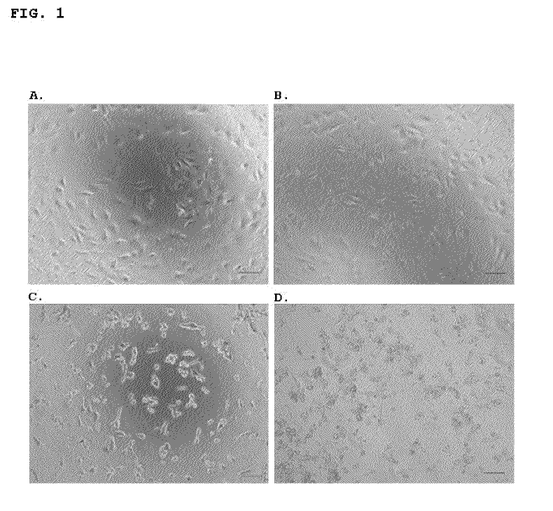

For a cyprosin concentration of 100 μg/mL the differences between the non-tumour FHs74 Int cells and HCT tumour cells became significant and can be visualized in FIG. 1.

In general, only concentrations of cyprosin preparation ranging between 1000 μg/mL e 100 μg/mL induced differences on the morphology of the different cell lines. The highest concentration (1000 μg/mL) induced lysis in all cell line populations (tumour and non-tumour).

The enzyme preparation at a concentration of 100 μg/mL induced significant morphological alterations on all tumour cells that became longer and thinner with visible signs of lysis (FIG. 1).

In turn, the non-tumour FHs74 Int and Vero cells, submitted to the same 100 μg/mL enzyme preparation, did not show morphological alterations (FIG. 1).

Using the method described, no sign of toxicity of the enzyme preparation was observed on the different cell lines assayed for enzyme concentrations below 10 μg/mL.

After microscopic observation, all the plate wells were incubated for 1 h at 4° C., with 50 μl of a 50% (w/v) TCA solution (Fluka). The plates were then washed 5 times with distilled water.

After the last wash, the plates were dried off and 100 μl of freshly prepared 0.4% (w/v) SRB (Sigma) were added to each well.

The plates were incubated for 30 minutes at room temperature and protected from light.

The SRB stain was removed from the cells by washing five times with 250 μL of 1% acetic acid (Rieldel-de Haen).

Each plate well was then incubated with 200 μL of a 10 mM Trizma base (Fluka) solution, for 10 minutes, at room temperature, protected from light, under constant shaking. The cells were ruptured and the SRB-stained proteins were released.

The assay was finished by measuring the absorbance in order to evaluate the relative growth and cell viability upon exposure to the cyprosin preparation and the controls.

To calculate the IC₅₀ values, the incorporation of SRB in the cellular proteins (% SRB) was evaluated against the control cells following the equation (1) were SRB_{E} represents the absorbance mean for each concentration of enzymatic preparation, SRB_{B} the absorbance mean for the blank assays and SRB_{C} the absorbance mean for the control assays:

% SRB=(SRB_{E}−SRB_{B})/(SRB_{C}−SRB_{B})×100 (1)

The curves in the graphics of % SRB versus logarithm of enzyme concentration (μg/ml) were adjusted using the Hill function (2), determined by the biostatistics program Prism 5, for Windows (GraphPad Software), where the background and signal parameters are respectively 0% and 100%:

Y=Background+(Signal-Background)/(1+10^{(logIC}^{₅₀}^{-X)*Hill slope}) (2)

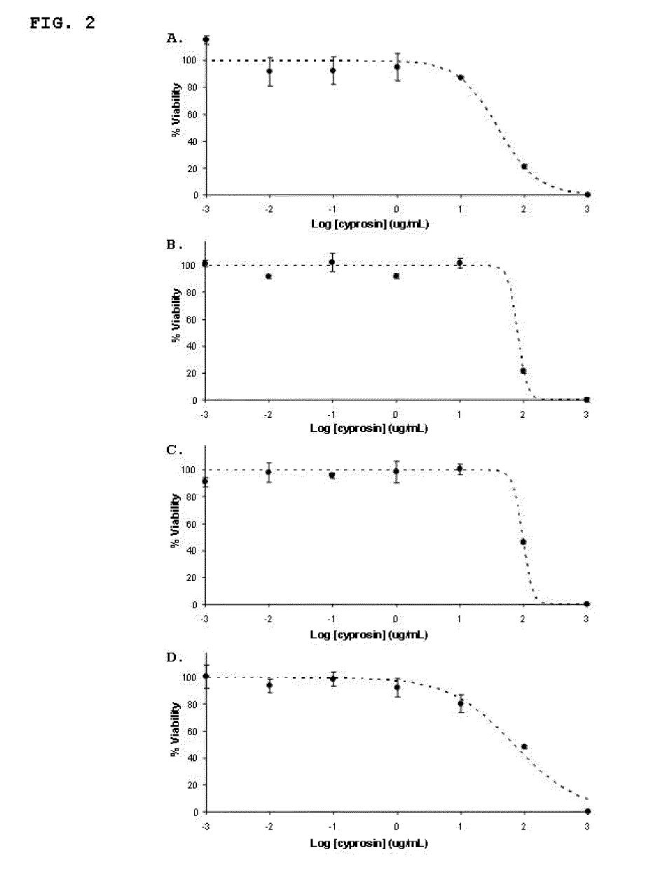

The graphical representation of the viability of cells stained with SRB, related to the logarithm of the cyprosin concentration (μg/ml), for each cell line, can be observed in FIG. 2. The values of the corresponding IC₅₀ are summarized in Table IV:

TABLE IV
IC₅₀ values obtained using the biostatistics
program Prism 5, for Windows (GraphPad Software), based on
the absorbance values obtained for each tumour and non-tumour
cell line.

| | | IC₅₀ |
|---|---|---|
| Tumour Cell Lines | TE671 | 97.54 μg/mL |
| | HT1080 | 81.09 μg/mL |
| | Hela | 69.73 μg/mL |
| | HCT116 | 38.59 μg/mL |
| Non-Tumour Cell Lines | Vero | 617.8 μg/mL |
| | FHs74 Int | 118.5 μg/mL |

For the studied tumour cell lines, it was observed that HCT116 cells are the most sensitive to the antitumour effect of enzyme preparation, while TE671 cells are the most resistant. For the non-tumour cell lines, it was observed that FHs74 Int cells are more sensitive than Vero cells.

The fact that tumour cell lines are consistently more susceptible to the cyprosin preparation, which can be demonstrated by their IC₅₀ values (five times lower in absolute terms than those obtained for non-tumour cells) is coherent with the morphological observations.

In general, these results represent a tumour cell-specific lethal effect of the native enzyme purified from dried flowers of Cynara cardunculus when compared to non-tumour cells submitted to the same concentrations of cyprosin preparations.

The results reported show that the potential antitumour cytotoxic effect of the native cyprosin preparation occurs at concentrations up to 1000 μg/ml.

### Example II

Antitumour activity of a preparation of recombinant cyprosin, containing the two structural chains: N-terminal chain (consisting of the N-terminal pro-peptide and the mature N-terminal peptide), and the C-terminal chain (consisting of the mature C-terminal peptide), isolated and purified from the culture medium of a Saccharomyces cerevisiae strain transformed with the CYPRO11 gene.

The cyprosin preparation was obtained from the supernatant from a culture of Saccharomyces cerevisiae strain (BJ1991), transformed with the CYPRO11 gene coding for cyprosin as previously described (Pais et al., 2000). The antitumour activity of the enzyme preparation was tested on a carcinoma-derived human tumour epithelial cell line (HCT116, ATCC CCL-247), as well as on a non-tumour cell line consisting of epithelial cells from human intestine (FHs74 Int, ATCC CCL-241).

The tumour cell line HCT116 was inoculated on basal medium DMEM (Cambrex), supplemented with foetal bovine serum (FBS—Gibco). The final concentrations of glucose (Sigma) and L-glutamine (Sigma) were 4.5 g/L and 6.0 mM, respectively.

The medium was supplemented with 1% Penicillin/Streptomycin (Gibco).

The non-tumour cell line FHs74 Int was inoculated on basal medium Hybricare (ATCC; Cat. 46-X), supplemented with 10% foetal bovine serum (FBS—Gibco); 2.10 g/L sodium bicarbonate (NaHCO₃) (Sigma); 2.0 mM L-glutamine (Sigma), and 30 ng/mL Epidermal Growth Factor (EGF—Sigma).

The medium was supplemented with 1% Penicillin/Streptomycin (Gibco).

The cells were propagated in a static culture system operated discontinuously. The cell concentration and viability were evaluated using the Trypan blue exclusion method.

The specific growth rate (t) and the doubling time of tumour and non-tumour cell lines, HCT116 e FHs74 Int respectively, are presented in Table III above (Example I).

Like in Example I, the morphological analysis of cells was performed by optical microscopy and the determination of IC₅₀ was done using the sulforhodamine B (SRB) method.

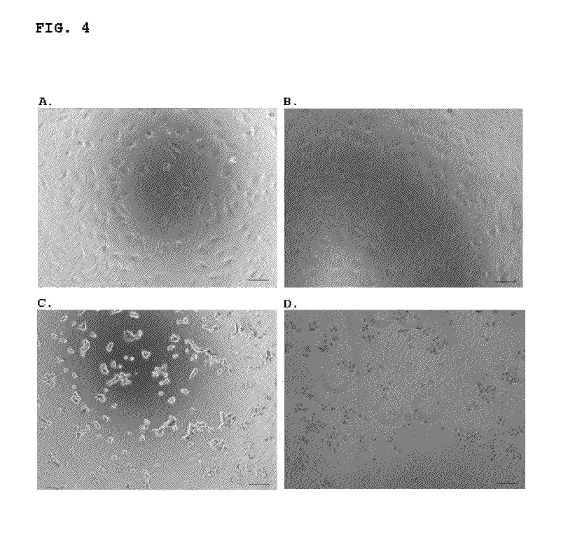

The results of the morphological analysis for cells treated with a 100 μg/mL of enzyme preparation are presented in FIG. 4. Contrasting with the non-tumour cell line FHs74 Int, which is not affected by the addition of recombinant cyprosin preparation, the HCT cells present clear evidence of lyses 48 h after addition of the enzyme preparation.

As in example I, the IC₅₀ parameters were determined for both cultures after the morphological study.

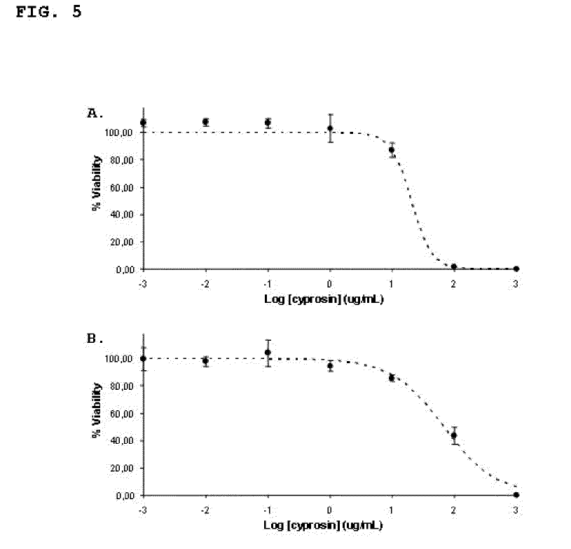

The percent cell viability variation of cells stained with SRB related to the logarithm of cyprosin concentration (μg/ml), for each cultured cell line, is represented in FIG. 5

The values of IC₅₀ were 20.51 μg/mL for the tumour cell line HCT116 and 70.50 μg/mL for FHs74 Int cell line indicating a three-fold higher susceptibility of the tumour cell line to the recombinant cyprosin than that observed with the control non-tumour cell line FHs74 Int.

The results also show a higher lethal effect of the recombinant cyprosin preparation (consistently lower IC₅₀ values) when compared to the natural cyprosin preparation.

The results suggest that the potential antitumour cytotoxic effect of the recombinant cyprosin preparation occurs at enzyme concentrations up to 100 μg/ml.

### REFERENCES

Beaujouin M., Baghdiguian S., Glondu-Lassis M., Berchem G. and E. Liaudet-Coopman (2006). Overexpression of both catalitically active and -inactive cathepsin D by cancer cells enhances apoptosis-dependent chemo-sensitivity. Oncogene 25:1967-1973.
Beers E. P., Bonnie J. W. and C. Zhao (2000). Plant proteolytic enzymes: possible roles during programmed cell death. Plant Mol. Biol. 44:399-415.
Bento I., Coelho R., Frazao C., Costa J., Faro C., Verissimo P., Pires E., Cooper J., Dauter Z., Wilson K. and M. A. Carrondo (1998). Crystallisation, structure solution, and initial refinement of plant cardosin-A. Adv Exp Med. Biol. 436:445-52.
Bidere N., Lorenzo H. K., Carmona S., Laforge M., Harper F., Dumont C. and A. Senik (2003). Cathepsin D triggers Bax activation, resulting in selective apoptosis-inducing factor (AIF) relocation in T lymphocytes entering the early commitment phase to apoptosis. J. Biol. Chem. 33:31401-31411.
Brodelius P. E., Cordeiro M. C and M. S. Pais (1995). Aspartic proteinases (cyprosins) from Cynara cardunculus spp. Flavescens cv. cardoon; purification, characterisation, and tissue-specific expression. Adv. Exp. Med. Biol. 362:255-66.
Brodelius P. E., Cordeiro M., Mercke P., Domingos A., Clemente A. and M. S. Pais (1998). Molecular cloning of aspartic proteinases from flowers of Cynara cardunculus SUBSP. flavescens CV. cardoon and Centaurea calcitrapa. Adv Exp Med. Biol. 436:435-439.
Buchanan-Wollaston V. (1997). The molecular biology of leaf senescence. J. Exp. Bot. 48:181-199.
Cordeiro M. C., Xue Z. T., Pietrzak M., Pais M. S, and P. E. Brodelius (1994). Isolation and characterization of a cDNA from flowers of Cynara cardunculus encoding cyprosin (an aspartic proteinase) and its use to study the organ-specific expression of cyprosin. Plant Mol. Biol. 24:733-741.
Cordeiro M. C., Xue Z. T., Pietrzak M., Pais M. S, and P. E. Brodelius (1995). Plant aspartic proteinases from Cynara cardunculus spp. flavescens cv. cardoon; nucleotide sequence of a cDNA encoding cyprosin and its organ-specific expression. Adv. Exp. Med. Biol. 362:367-72
Cordeiro M. C., Lowther T., Dunn B. M., Guruprasad K., Blundell T., Pais M. S, and P. E. Brodelius (1998). Substrate specificity and molecular modelling of aspartic proteinases (Cyprosins) from flowers of Cynara cardunculus subsp. Flavescens cv. Cardoon. Aspartic proteinases 436:473-479.
Costa J., Ashford D. A, Nimtz M., Bento I., Frazdo C., Esteves C. L., Faro C. J., Kervinen J., Pires E., Verissimo P., Wlodawer A. and M. A. Carrondo (1997). The glycosilation of aspartic proteinases from barley (Hordeum vulgare L.) and cardoon (Cynara cardunculus L.). Eur. J. Biochem. 243:695-700.
Deiss L. P., Galinka H., Berissi H., Cohen O. and A. Kimchi (1996). Cathepsin D protease mediates programmed cell death induced by interferon-γ, FAS/APO-1 and TNF-α. EMBO J. 15:3861-3870.
Dunn B. M. (2002). Structure and mechanism of the pepsin-like family of aspartic peptidases. Chem. Rev. 102:4431-4458.
Faro C., Verissimo P., Lin Y., Tang J. and E. Pires (1995). Cardosin A and B, aspartic proteases from the flowers of cardoon. Adv. Exp. Med. Biol. 362:373-7.
Faro C., Ramalho-Santos M., Vieira M., Mendes A., Simaes I., Andrade R., Veríssimo P., Lin X., Tang J and E. Pires (1999) Cloning and Characterization of cDNA encoding Cardosin A, an RGD-containing Plant Aspartic Proteinase. J. Biol. Chem. 274:28724-28729.
Frazão C., Bento I., Costa J., Soares J. M., Verissimo P., Faro C., Pires E., Cooper J. and M. A. Carrondo (1999). Crystal structure of cardosin A, a glycosylated and Arg-Gly-Asp-containing aspartic proteinase from the flowers of Cynara cardunculus. J. Biol. Chem. 274:27694-27701.
Glathe S., Kervinen J., Nimtz M., Li G. H., Tobin G. J., Copeland T. D., Ashford D. A., Wlodawer A. and J. Costa (1998) Transport and activation of the vacuolar aspartic proteinase phytepsin in barley (Hordeum vulgare L.). J. Biol. Chem. 273:31230-31236.
Haendeler J., Popp R., Goy C., Tischler V., Zeiher A. M. and S. Dimmeler (2005). Cathepsin D and H₂O₂ simulate degradation of thioredoxin-1: implication for endothelial cell apoptosis. J. Biol. Chem. 280:42945-42951.
Heimgartner U., Pietrzak M., Geerstsen R., Brodelius A. C., Figueiredo A. C. da Silva and M. S. S. Pais (1989). Purification and partial characterization of milk clotting proteases from flowers of Cynara cardunculus. Phytochemistry 29:1405-1410.
Iacobuzio-Donahue C., Shuja S., Cai J., Peng P., Willett J. and M. J. Murnane (2004) Cathepsin D protein levels in colorectal tumors: divergent expression patterns suggest complex regulation and function. Int. J. Oncol. 3:473-485.
Kervinen J., Tobin G. J., Costa J., Waugh D. S., Wlodawer, A. and A. Zdanov (1999). Crystal structure of plant aspartic proteinase prophytepsin: inactivation and vacuolar targeting. EMBO J. 18:3947-3955.
Monks A., Scudiero D., Skehan P., Shoemaker R., Paull K., Vistica D., Hose C., Langley J., Cronise P., Vaigro-Wolff A., Gray-Goodrich M., Campbell H., Mayo J. and M. Boyd (1991). Feasibility of high-flux anticancer drug screen using a diverse panel of cultured human tumour cell lines. J. Nat. Can. Inst. Vol. 83, No 11.
Panavas T., Pikla A., Reid P. D., Rubinstein B. and E. L. Walker (1999). Identification of senescense-associated genes from daylily petals. Plant Mol. Biol. 40:237-248.
Pais M. S. S., Conceição F. C. C. and J. Rudy (2000). Production by yeasts of aspartic proteinases from pant origin with sheep's, cow's, goat's milk, etc. clotting and proteolytic activity. WO/2000/075283.
Piwnica D., Touraine P., Struman I., Tabruyn S., Bolbach G., Clapp C., Matial J. A., Kelly P. A. and V. Goffin (2004). Cathepsin D processes human prolactin into multiple 16K-like N-terminal fragments: study of their antiangiogenic properties and physiological relevance. Mol. Endocrinol. 10:2522-2542.
Ramalho-Santos M., Pissarra J., Verissimo P., Pereira S., Salema R., Pires E. and C. J. Faro. (1997). Cardosin A, an abundant aspartic proteinase, accumulates in protein storage vacuoles in the stigmatic papillae of Cynara cardunculus L. Planta, 203:204-12.
Ramalho-Santos M., Verissimo P., Cortes L., Samyn B., Van Beeumen J. and E. Pires (1998). Identification and proteolytic processing of procardosin A. Eur. J. Biochem. 255:133-138.
Rawlings N. D., Morton F. R. and A. J. Barrett (2006). MEROPS: the peptidase database. Nucleic Acids Res 34:D270-D272.
Runeberg-Roos P., Tormakangas, K. and A. Ostman (1991). Primary structure of a barley-grain aspartic proteinase: a plant aspartic proteinase resembling mammalian Cathepsin D. Eur. J. Biochem. 202:1021-1027.
Ruoslahti E. (1996). RGD and other recognition sequences for integrins. Annu. Rev. Cell. Dev. Biol. 12:697-715.
Skehan P., Storeng R., Scudiero D., Monks A., McMahon J., Vistica D., Warren J. T., Bokesch H., Kenney S, and M. R. Boyd (1990). Evaluation of colorimetric protein and biomass stains for assaying drug effects upon human tumour cell lines. Proc. Amer. Assoc. Cancer Res. 13:1107-1112.
Wang Z., Liang R., Huang G. S., Piao Y., Zhang Y. Q., Wang A. Q., Dong B. X., Feng J. L., Yang G. R. and Y. Guo (2006). Glucosamine sulfate-induced apoptosis in chronic myelogenous leukemia K562 cells is associated with translocation of cathepsin D and down regulation of Bcl-xL. Apoptosis 10:1851-60.
Wu G. S., Saftig P., Peters C. and W. S. El-Deiry (1998). Potential role for Cathepsin D in p53-dependent tumor suppression and chemosensitivity. Oncogene 17:2177-2183.
Verissimo P., Faro C., Moir A. J., Lin Y., Tang J. and E. Pires (1996). Purification, characterization and partial amino acid sequencing of two new aspartic proteinases from fresh flowers of Cynara cardunculus L. Eur. J. Biochem. 235:762-8.

1-20. (canceled)

21. A phytepsin for use as a medicament.

22. Phytepsin according to claim 21 for use in the treatment of cancer.

23. Phytepsin according to claim 22 for use in the treatment of colon-rectal, small intestine, uterine cervix, ovarian, prostate, stomach, breast, bladder, lymph, sarcoma, pancreas, melanoma, glyoma, neuroblastoma, lung, mouth, head and neck, liver, cervical, and haematological cancers.

24. Phytepsin according to claim 23 wherein the protein is cyprosin.

25. Phytepsin according to claim 24 wherein the cyprosin is extracted, with or without purification, from a natural source, specifically, but not limited to, from Cynara cardunculus.

26. Phytepsin according to claim 24 wherein the cyprosin is recombinant produced from heterologous sources such as a microorganism or a genetically modified cell line.

27. Phytepsin according to claim 26 wherein the cyprosin recombinant protein is obtained from a recombinant protein expressing system such as, but not restricted to, Saccharomyces cerevisiae or Escherichia coli.

28. Phytepsin according to claim 26 wherein the cyprosin recombinant protein is obtained from a cell culture of genetically modified cell lines, such as, but not restricted to, cell lines derived from insects and mammals, namely human cell lines.

29. Phytepsin according to claim 28 wherein the cyprosin comprises all translation products of the cyprosin transcript/s, including peptide species resulting from either post-transcriptional or post-translational processing/maturation of cyprosin transcripts and/or polypeptides, respectively.

30. Phytepsin according to claim 28 wherein the cyprosin consists of the cyprosin pre-propeptide and/or the cyprosin propeptide containing the N-terminal and/or the mature N-terminal subunit/peptide chain, and/or the cyprosin propeptide containing the C-terminal and/or the PSI domain specific of plant phytepsins and/or the mature C-terminal subunit/peptide chain and/or the isolated polypeptide containing the PSI domain and/or any other secondary product derived from processing or degradation of the initial pre-propeptide.

31. Phytepsin according to claim 30 wherein the cyprosin consists of one or more peptides with amino acid sequence/s that can be deduced from the cyprosin pre-propeptide amino acid sequence, either resulting from a modified DNA sequence, and/or resulting from polypeptide degradation, and/or resulting from enzymatic digestion of the pre-propeptide, and/or resulting from its natural processing mechanism, and/or obtained by chemical synthesis.

32. Pharmaceutical compositions containing the phytepsin of claim 21.

33. Pharmaceutical compositions according to claim 32 additionally containing pharmaceutically acceptable excipients, carriers, additives, diluents, solvents, filters, lubrificants, stabilising compounds and/or adjuvants.

34. Pharmaceutical compositions according to claim 32 containing the active peptide species conjugated with immune system-interacting elements, such as antibodies or any of their chains/subunits or fragments, and/or immuno-stimulants, such as antigens, T-lymphocytes with cytotoxic activity, and/or dendritic cells.

35. Pharmaceutical compositions according to claims 32 wherein the pharmaceutical compositions are administered in combination, conjugation, or insertion with respect to transporting molecules.

36. Pharmaceutical compositions according to claims 32 wherein the pharmaceutical compositions are administered within vehicles such as, but not restricted to, encapsulating nanoparticles.

37. Pharmaceutical compositions according to claim 32 wherein the pharmaceutical compositions are administered in either a systemic or localized fashion, intravenously, orally, or in any other fashion.

38. Pharmaceutical compositions according to claim 32 wherein the pharmaceutical compositions are administered to animals, preferentially mammals, namely humans.

39. Pharmaceutical compositions according to claim 32 characterised by presenting antitumour activity in vitro in cell lines such as, but not restricted to, a human epithelial cell line derived from colon carcinoma (HCT), a human epithelial cell line derived from an adenocarcinoma (HeLa), a human cell line derived from a fibrosarcoma (HT), and an human epithelial cell line derived from a medulloblastoma (TE).

40. Pharmaceutical compositions according to claim 32 characterized by inhibiting growth in 50% of tumour cell lines such as, but not restricted to, a human epithelial cell line derived from colon carcinoma (HCT), a human epithelial cell line derived from an adenocarcinoma (HeLa), a human cell line derived from a fibrosarcoma (HT), and a human epithelial cell line derived from a medulloblastoma (TE), containing cyprosin preparation concentrations ranging from 1 to 100 μg/ml.

41. Pharmaceutical compositions according to claim 32 characterised by inhibiting in 50% the growth of human tumour cell lines at concentrations of cyprosin preparations ranging from 0.001 to 1 μg/ml.

42. Pharmaceutical compositions according to claim 32 wherein the pharmaceutical compositions are used to restore physiological conditions or human pathologies, namely, but not restricted to, high blood pressure, retroviral infection, haemoglobin degradation and digestive problems.

43. Use of a phytepsin protein for the manufacture of a medicament for the treatment of cancer.

44. Use of a phytepsin protein according to claim 43 for the treatment of colon-rectal, small intestine, uterine cervix, ovarian, prostate, stomach, breast, bladder, lymph, sarcoma, pancreas, melanoma, glyoma, neuroblastoma, lung, mouth, head and neck, liver, cervical, and haematological cancers.

45. Use of a phytepsin protein according to claim 43 wherein the protein is cyprosin.
